(19) United States
(12) Patent Application Publication
(10) Pub. No.: US 2004/0180094 A1
(43) Pub. Date: Sep. 16, 2004

(54) Activation agents on the surface of encapsulation vesicles

(75) Inventor: Timothy H. Joyce, Sunnyvale, CA (US)

Correspondence Address: ORRICK, HERRINGTON & SUTCLIFFE, LLP, 4 PARK PLAZA, SUITE 1600, IRVINE, CA, 92614-2558, US

(73) Assignee: Hemolytics, Inc.

(21) Appl. No.: 10/387,301

(22) Filed: Mar. 11, 2003

(51) Int. Cl. A61K 9/50; C07K 17/00; C07K 14/00; C07K 1/00; A61K 9/16

(52) U.S. Cl. 424/490; 530/350

(57) ABSTRACT

The present invention relates to a composition and method for therapeutic treatment of humans and other mammals. The present invention may address drug resistance problems in vivo. The therapeutic composition of the present invention comprises an encapsulation vesicle, an activation agent such as a pore forming agent on the surface of the encapsulation vesicle and an optional targeting ligand. The targeting ligand may be attached to either the activation agent or the encapsulation vesicle. The encapsulation vesicle may contain a bioactive agent that may be released to the inside of a diseased cell. The activation agent or pore forming agent is activated by an activation condition.

The method for therapeutic treatment includes contacting a cell membrane with a therapeutic composition that has an encapsulation vesicle and an activation agent such as a pore forming agent on the surface of the encapsulation vesicle and allowing the cell membrane to incorporate the therapeutic composition so that the activation agent or pore forming agent of the therapeutic composition may be activated by an activation condition.

### FIELD OF THE INVENTION

The present invention is directed generally toward a composition and method for therapeutic treatment and more particularly toward targeted encapsulation vesicles that contain activation agents on their surface that may be activated at will.

### BACKGROUND OF THE INVENTION

A number of different types of therapeutic agents and compositions are described in the literature for treating diseases such as anthrax, HIV infection and cancer. Standard drug therapies, however, suffer from a number of important limitations including the development of drug resistance over time, high dose of agent required, toxicity toward normal cells, drug inactivation and loss of drug selectivity. High levels of drugs or drug cocktails continue to be the only means for addressing some of these problems.

Drug encapsulation of bioactive compounds has been extensively studied in a variety of systems in attempts to address some of these problems. Drug delivery systems provide the ability to deliver high levels of drug load while lessening immunological response. Bioavailability refers to the presence of drug molecules where they are needed most in the body and where they will do the most good. Therapeutics and drug delivery systems have centered on maximizing bioavailability both over a period of time and specific locations in the body. However, most pharmaceuticals today also suffer from poor bioavailability. Therefore, there is also an ongoing need to improve bioavailability in therapeutic treatments for a variety of diseases.

Nanotechnology and nanoscience are very useful in developing entirely new schemes for increasing drug delivery and bioavailability. Nanotechnology and nanostructure in particular are of interest because of their ability to self-assemble. Whitesides et al., Science (1991) 254:1312-1319. Bates, Science (1991) 251:898-905; Gunther & Stupp, Langmuir (2001) 17:6530-6539; Hulteen et al., J. Am. Chem. Soc. (1998) 120:6603-6604; Moore and Stupp., J. Am. Chem. Soc. (1992): 9-14; Muthukumar et al, Science (1997) 277:1225-1232; Stupp et al., Science (1997) 276:384-389; Stupp et al., Science (1993) 259:59-63; and Zubarev et al., Science (1999) 283:523-526. These structures provide for efficient and effective fabrication that is defect free. There are already a number of biologically based systems that self-assemble. For instance, T4 phage particles, and the transmembrane toxin α-hemolysin that forms a heptamer upon oligomeric association with cell membranes.

Furthermore, a number of technologies are under development to address some of the more important therapeutic problems. For instance, molecules can be encapsulated in nanoscale cavities inside polymers. These polymers can then be swallowed as a tablet and the polymeric structure can be activated in the digestive tract to release the enclosed drug over time. These time-released methods can provide some advantages over standard drug therapies. Another simpler scheme is to grind solid drugs or drug material into fine particles at the nanoscale. These fine powders can increase surface area and reactivity. Other drug delivery systems have been developed using cholesterol and liposome structures to encapsulate and deliver soluble proteins such as cytokines. Interesting combinations of smart materials have also been developed for delivering drugs under a triggered response. These therapeutic techniques include placing a drug molecule within the body in an inactive form that “wakes up” under an endogenous or exogenous stimulus. Some simple examples of this type of technology include an antacid enclosed in a coating of a polymer that dissolves in acid conditions. These techniques, however, suffer from the limitation that none of them address drug resistance problems that arise over-time.

Photodynamic therapy is a more advance technique for therapeutic treatment that may be used to “wake-up” an inactive drug and improve bioavailability in a patient. For instance, light with long wavelengths can actually pass through biological tissues without excessive scattering and can be used to effect processes within the human body. In photodynamic therapy a particle is placed within the body and is illuminated by an external source. The external source may include a laser, light or lamp. The light is absorbed by the particle and can be used to heat, activate or chemically alter a particle that has been placed in the body. Present photodynamic therapies include the use of quantum dots or molecular dots. This technique has the advantage over traditional chemotherapy treatments and drug cocktails in that the therapy does not leave a “toxic trail” of highly reactive or toxic molecules in the body. However, this technology suffers from the limitation that it is not capable of being targeted to diseased cells.

As such, there is a need for therapeutic treatments that increase bioavailability, address drug resistance problems, and which can be easily assembled, activated and targeted with low levels of toxicity in the patient. In addition, since many drug resistance problems develop internal to the cell membrane or inside a pathogenic cell there is a need for treatments that work on the surface of the cell and that do not allow for drug inactivation or development of drug resistance. The present invention satisfies these and other needs.

### SUMMARY OF THE INVENTION

The present invention relates to a composition of matter and method for therapeutic treatment of humans and other mammals. The present invention may address drug resistance problems in vivo. Basically, the therapeutic composition of the present invention comprises an encapsulation vesicle, an activation agent such as a pore forming agent on the surface of the encapsulation vesicle and an optional targeting ligand.

The encapsulation vesicle must be capable of allowing an activation agent such as a pore forming agent on its surface. The encapsulation vesicle may optionally allow the attachment of a targeting ligand and/or enclose a bioactive agent.

An important component of the invention is the activation agent. The activation agent is capable of being activated by an activation condition. In certain instances the activation agent may be self assembling, but this is not required. It is important to the invention that the activation agent be associated with the surface of the encapsulation vesicle. This provides the ability to avoid drug resistance problems that arise internal to a cell and allows for activation of the activation agent by the activation condition. The activation agent may comprise a pore forming agent.

The invention also provides a method for therapeutic treatment using the composition of the invention. The method for therapeutic treatment comprises contacting a cell membrane with a therapeutic composition that comprises an encapsulation vesicle and an activation agent on the surface of the encapsulation vesicle and allowing the cell membrane to incorporate the therapeutic composition so that the activation agent of the therapeutic composition may be activated.

### BRIEF DESCRIPTION OF THE DRAWINGS

FIG. 1 shows a photodynamic pore forming agent and its method of activation.

FIG. 2. shows the one-pot synthesis steps of BNPA.

FIG. 3. shows the formation of CNB thioethers and their proposed photochemistry.

FIG. 4. shows the method of therapeutic treatment of the present invention.

FIG. 5. shows the application of the photodynamic pore forming agents and how they may be applied in a topical therapeutic treatment.

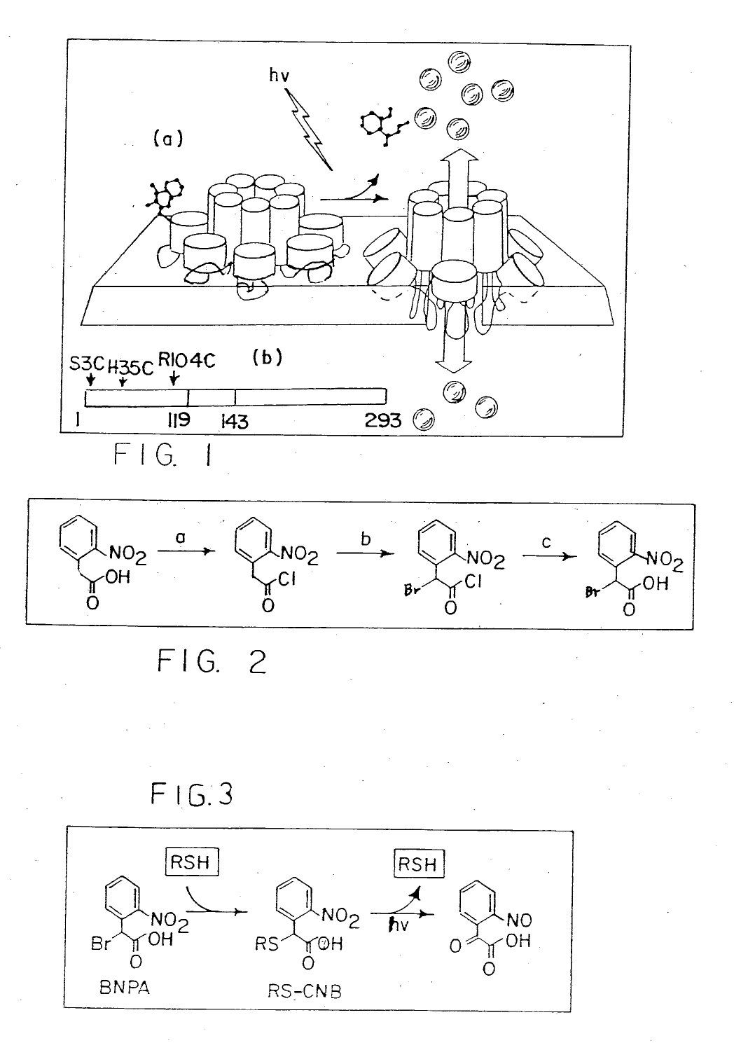

### DESCRIPTION OF THE SPECIFIC EMBODIMENTS

Methods for therapeutic treatment are provided. In the described methods the therapeutic composition may be used to contact a cell membrane. The cell membranes may be in vitro or in vivo and include both pathogenic and nonpathogenic cells unless clearly stipulated otherwise.

Before describing the invention in further detail, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a”, “an” and “the” include plural reference unless the context clearly dictates otherwise.

“Activate” or “activate by an activation condition” refers to the application of physical, chemical or biochemical conditions or processes that will cause a pore forming agent to open, close, open and close, open or close, degrade, release a bioactive agent through or by the pore forming agent, release one or more molecules that may be photodynamically activated or activated by other activating conditions. For instance, a pore forming agent may be activated by an external light source or laser to open and release a photodynamic bioactive agent.

“αHL-K8A” refers to a mutant hemolysin protein produced by replacing the lysine (K) at position 8 in the amino acid sequence with arginine (A).

“αHL-H5M ” refers to a mutant hemolysin protein produced by replacing the histidine (H) at position 5 in the amino acid sequence with methionine (M).

αHL(1-172●132-293) refers to a particular mutant α-hemolysin protein that has been produced using recombinant DNA techniques.

R104C refers to the replacement of arginine (R) 104 in the α-hemolysin protein with cysteine (C).

K168C, refers to the replacement of lysine (K) 168 in the α-hemolysin protein with cysteine (C).

D183C refers to the replacement of aspartate (D) 183in the α-hemolysin protein with cysteine (C).

E11C refers to the replacement of glutamate (E) 11 in the α-hemolysin protein with cysteine (C).

“Bioactive agent” refers to a substance that may be used in connection with an application that is therapeutic or diagnostic, such as, for example, in methods for diagnosing the presence or absence of a disease in a patient and/or methods for treatment of a disease in a patient. The term also refers to a substance that is capable of exerting a biological effect in vitro or in vivo. The bioactive agents may be neutral, positively or negatively charged. Exemplary bioactive agents include for example prodrugs, targeting ligands, diagnostic agents, pharmaceutical agents, drugs, synthetic organic molecules, proteins, peptides, vitamins, steroids, steroid analogs and genetic material.

“Biocompatible” refers to materials that are generally not injurious to biological functions and which will not result in any degree of unacceptable toxicity, including allergenic responses and diseased states.

“Biomolecule” refers to molecules derived from a biological organism or source. For example, biomolecules may include and not be limited to proteins, peptides, amino acids, nucleotides, nucleosides, polynucleotides, carbohydrates, lipids, sphingolipids, deoxyribonucleic acid (DNA), ribonucleic acid (RNA), tRNA, mRNA, derivatives or these materials, collagen, fibrinogen, antibodies and other well known materials from biological organisms.

“Carrier” refers to a pharmaceutically acceptable vehicle, which is a nonpolar, hydrophobic solvent, and which may serve as a reconstituting medium. The carrier may be aqueous based or organic based. Carriers include, inter alia, lipids, proteins, polyscaccharides, sugars, polymers, copolymers, and acrylates.

“Cell” refers to any one of the minute protoplasmic masses which make up organized tissue, comprising a mass of protoplasm surrounded by a membrane, including nucleated and unnucleated cells and organelles.

“Cell membrane” refers the commonly described lipid based exterior boundary of a cell. The cell membrane may or may not comprise proteins or receptors.

“Diseased cell”, “pathogenic cell” or “pathological cell” refers to any cell that fails to operate in its naturally occurring condition or normal biochemical fashion. These cells should be capable of causing disease. For instance, the word shall include cells that are subject to uncontrolled growth, cellular mutation, metastasis or infection. The term shall also include cells that have been infected by a foreign virus or viral particle, bacteria, bacterial exotoxins or endotoxins, prions, or other similar type living or non-living materials. The term may in particularly refer to cancer cells or cells infected by the polio virus, rhinovirus, piconavirus, influenza virus, or a retrovirus such as the human immunodeficiency virus (HIV).

“Fusion” refers to the joining together of components to form a single contiguous component. For instance, when two cell membranes contact each other the lipids, proteins or other cellular materials re-associate and/or reorganize to form a single contiguous membrane.

“Genetic material” or “therapeutic charge” refers to nucleotides and polynucleotides, including deoxyribonucleic acids (DNA) and ribonucleic acid (RNA). The genetic material may be made by synthetic chemical methodology, may be naturally occurring, or may be made by commonly known recombinant DNA techniques. The nucleotides, DNA, and RNA may contain one or more modified bases or base pairs, or unnatural nucleotides or biomolecules.

“Incorporate” refers to one or more processes for taking up a component, agent, material, cell membrane or biomolecule. Incorporation processes may include invagination, phagocytosis, endocytosis, exocytosis or fusion processes. These processes may or may not further include one or more clathrate coated pits or receptors.

“Intracellular” or “intracellularly” refers to the area within the plasma membrane of a cell, including the protoplasm, cytoplasm and/or nucleoplasm.

“Intracellular delivery” refers to delivery of a bioactive agent, such as a targeting ligand and/or prodrug or drug, into the area within the plasma membrane of the cell.

“Lipid” refers to a naturally occurring, synthetic or semi-synthetic (i.e. modified natural) compound that is generally amphipathic. The lipids typically comprise a hydrophilic component and a hydrophobic component. Exemplary lipids include, for example, fatty acids, neutral fats, phosphatides, oils, glycolipids, surface active agents (surfactants), aliphatic alcohols, waxes, terpenes and steriods. The phrase semi-synthetic (or modified natural) denotes a natural compound that has been chemically modified in some fashion.

“Liposome” refers to a generally spherical or spheroidal cluster or aggregate of amphipathic compounds, including lipid compounds, typically in the form of one or more concentric layers, for example bilayers. They may also be referred to as lipid vesicles. The liposome may be formulated, for example, from ionic lipids and/or non-ionic lipids. Liposomes formulated from non-ionic lipids may be referred to as niosomes.

“Nanoerythrosome” refers to a vesicle structure that is derived from erythrocytes and substantially free of hemoglobin. These vesicles have a size of less than about 1 micrometer to about 0.1 micrometer and are substantially spherical or spheroidal. The term refers to any bioactive agent carrier described in U.S. Pat. No. 5,653,999 and associated patents or patent applications (herein incorporated by reference in their entirety).

“Nanocomposites” refers to composite structures whose characteristic dimensions are found on the nanoscale. An example is the suspension of carbon nanotubes in a soft plastic host.

“Nanodot” refers to nanoparticles that consist of homogenous material, especially those that are almost spherical or cubical in shape.

“Nanoparticle” refers to any material that can be made, ground or produced on the nanoscale.

“Nanopore” refers to a pore or passage through the structure that has a nanoscale inner diameter, where the inner diameter ranges, in many embodiments from about 0.1 to about 400 nanometers, such as from 10 to 30 nanometers, or from 5 to 10 nanometers.

“Nanorod” refers to nanostructures that are shaped like long sticks or dowels, with a diameter in the nanoscale and a length very much longer.

“Nanoscale” refers to phenomena that occur on the length scale between 1 and 100 nanometers.

“Nanostructure” refers to structures whose characteristic variation in design length is on the nanoscale.

“Nanowire” refers to nanorods that can conduct electricity.

“Patient” refers to animals, including mammals, preferably humans.

“Photodynamic” or “photodynamic therapy” refers to therapy that is promoted by light. The term includes molecules that are photogenerated. For instance, a pore forming agent may be activated to open and dynamically release an encapsulated drug or photoactivatable or excitable compound. In addition, a pore forming agent may open and/or compositionally degrade and/or release molecules that may be photo-excitable (i.e. the list includes and is not limited to quantum dots, nanodots, chromophores, fluorophores, dyes, suicide inhibitors etc . . . ). The term also includes molecules or therapies that are promoted by light and which also depend on the excited state dynamics of the molecules involved. For instance, photodynamic therapy delivers photosensitive chemicals called porphyrins intravenously. These molecules then collect rapidly in proliferating cells and when exposed to light, initiate a cascade of molecular reactions that can destroy cells or tissues they compose. Some targets for this type of therapy often include, but are not limited to abnormal blood vessels, retinas of people with age related macular degeneration, cancerous tumors and atherosclerotic plaques in coronary arteries.

“Polymer” refers to molecules formed from chemical union of two or more repeating units. Accordingly, included within the term “polymer” may be, for example, dimers, trimers and oligomers. The polymer may be synthetic, naturally occurring or semi-synthetic. The term may refer to molecules that comprise 10 or more repeating units.

“Protein” refers to molecules comprising essentially alpha-amino acids in peptide linkages. Included within the term “protein” are globular proteins such as albumins, globulins and histones, fibrous proteins such as collagens, elastins and keratins. Also included within the term are compound proteins, wherein a protein molecule is united with a non-protein molecule, such as nucleoproteins, mucoproteins, lipoproteins and metalloproteins. The proteins may be naturally occurring, synthetic or semi-synthetic.

“Receptor” refers to a molecular structure within a cell or on the surface of a cell that is generally characterized by the selective binding of a specific substance. Exemplary receptors include cell surface receptors for peptide hormones, neurotransmitters, antigens, complement fragments, immunoglobulins and cytoplasmic receptors for steroid hormones.

“Region of a patient” refers to a particular area or portion of the patient and in some instances to regions throughout the entire patient. Examples of such regions include the eye, gastrointestinal regions, cardiovascular regions (including myocardial tissue), circulatory system, bladder, mucosa, renal region, vascular tissues, as well as disease tissue such as cancerous tissue including prostate, breast, gallbladder, and liver. The term includes, for example, areas to be targeted by a drug delivery device or a bioactive agent. The term refers to both topical and internal organs and tissues. The phrase “vascular” or “vasculature” denotes blood vessels (including arteries, veins, and the like). The phrase “gastrointestinal region” includes the region defined by the esophagus, stomach, small intestine, large intestine, and rectum. The phrase “renal region” denotes the region defined by the kidney and the vasculature that leads directly to and from the kidney and includes the abdominal aorta.

“Region to be targeted” or “targeted region” refers to a region where delivery of a therapeutic is desired.

“Solid-state” or “solid state material” refers to materials that are not biological, biologically based or biological in origin. Such materials may include organic chemicals, synthetic fibers or materials, polymers, plastics, semiconductor materials, silica or silicon based substrates or materials, carbon based nanotubes, quantum dots, artificial bone cylinders, magnetic nanoparticles, suicide inhibitors, nanodots, nanostructures, or nanowires. These structures may be inserted into, comprise a portion of or be attached to the encapsulation vesicles or pore forming agents. In certain instance they may also comprise the pore forming agent. These materials should be capable of activation by an activation condition.

“Suicide inhibitor” refers to synthetic molecules that, upon reacting with an enzyme, produce a product that binds to the enzyme and, therefore, causes the enzyme not to function (to commit functional suicide).

“Surface” or “on the surface of the encapsulation vesicle” refers to being covalently or noncovalently attached to the exterior, associated with the exterior, embedded or partially embedded or forming a pore or channel through. For instance a pore forming agent on the surface of an encapsulation vesicle may be covalently or noncovalently attached to the exterior of the encapsulation vesicle, it may be embedded or partially embedded in the encapsulation vesicle, or it may create a channel or pore through the encapsulation vesicle. Channels or pores may allow for release of bioactive agents. Pore forming agents on the surface of an encapsulation vesicle should be capable of activation by an activation condition.

“Targeting ligand” or “target ligand” refers to any material or substance that may promote targeting of tissues and/or receptors in vivo or in vitro with the therapeutic compositions of the present invention. The targeting ligand may be synthetic, semi-synthetic, or naturally occurring. Materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steriods, steriod analogs, hormones, cofactors, bioactive agents, genetic material, including nucleotides, nucleosides, nucleotide acid constructs and polynucleotides.

“Therapeutic” refers to any pharmaceutical, drug or prophylactic agent which may be used in the treatment (including the prevention, diagnosis, alleviation, or cure) of a malady, affliction, disease or injury to a patient. Therapeutic includes contrast agents and dyes for visualization, Therapeutically useful peptides, polypeptides and polynucleotides may be included within the meaning of the term pharmaceutical or drug.

“Tissue” refers generally to specialized cells that may perform a particular function. The term refers to an individual cell or plurality or aggregate of cells, for example, membranes, blood or organs. The term also includes reference to an abnormal cell or plurality of abnormal cells. Exemplary tissues include myocardial tissue, including myocardial cells, membranous tissues, including endothelium and epithelium, laminae, connective tissue, including interstitial tissue, and tumors.

“Vesicle” or “encapsulation vesicle” refers to an entity that is generally characterized by the presence of one or more walls or membranes that form one or more internal voids. Vesicles may be formulated, for example, from a stabilizing material such as a lipid, including the various lipids described herein, a proteinaceous material, including the various proteins described herein, and a polymeric material, including the various polymeric materials described herein. As discussed herein, vesicles may also be formulated from carbohydrates, surfactants, and other stabilizing materials, as desired. The lipids, proteins, polymers and/or other vesicle forming stabilizing materials, may be natural, synthetic or semi-synthetic. Preferred vesicles are those which comprise walls or membranes formulated from lipids. The walls or membranes may be concentric or otherwise. The stabilizing compounds may be in the form of one or more monolayers or bilayers. In the case of more than one monlayer or bilayer, the monolayers or bilayers may be concentric. Stabilizing compounds may be used to form a unilamellar vesicle (comprised of one monolayer or bilayer), an oligolamellar vesicle (comprised of more than about three monolayers or bilayers). The walls or membranes of vesicles may be substantially solid (uniform), or referred to as, for example, liposomes, lipospheres, nanoliposomes, particles, micelles, bubbles, microbubbles, microspheres, nanospheres, nanostructures, microballoons, microcapsules, aerogels, clathrate bound vesicles, hexagonal/cubic/hexagonal II phase structures, and the like. The internal void of the vesicle may be filled with a wide variety of materials including, for example, water, oil, gases, gaseous precursors, liquids, fluorinated compounds or liquids, liquid perfluorocarbons, liquid perfluoroethers, therapeutics, bioactive agents, if desired, and/or other materials. The vesicles may also comprise a targeting ligand if desired.

“Vesicle stability” refers to the ability of vesicles to retain the gas, gaseous precursor and/or other bioactive agents entrapped therein after being exposed, for about one minute, to a pressure of about 100 millimeters (mm) of mercury (Hg). Vesicle stability is measure in percent (%), this being the fraction of the amount of gas which is originally trapped in the vesicle and which is retained after release of the pressure. Vesicle stability also includes “vesicle resilience” which is the ability of a vesicle to return to its original size after release of the pressure.

Where a range of values is provided, it is understood that each intervening value, to the tenth of a unit of the lower limit unless the context clearly dictates otherwise, between the upper and the lower limit of that range, and any other stated or intervening value in that stated range, is encompassed within the invention. The upper and lower limits of these smaller ranges are also encompassed within the invention, subject to any specifically excluded limit in the stated range. Where the stated range includes one or both of the limits, ranges including either or both of those included limits are also included in the invention.

Unless defined otherwise, all technical and scientific terms used herein have the same meaning as commonly understood to one of ordinary skill in the art to which this invention belongs. If for some reasons the usage or definitions herein shall be interpreted to differ from the commonly understand usage, then the definitions, herein, shall prevail. Although any methods, devices and materials similar or equivalent to those described herein can be used in the practice or testing of the invention, the methods, devices and materials are now described. Methods recited herein may be carrier out in any order of the recited events which is logically possible, as well as the recited order of events.

All patents and other references cited in this application infra and supra, are hereby incorporated by reference except as they may conflict with those of the present application (in which case the present application prevails).

In further describing the present invention, the therapeutic composition and methods of making the composition are first described in general detail. Then a few representative applications are provided. Subsequently, the method of therapeutic treatment using the therapeutic composition is then described and examples provided.

Activation Agents:

An important component of the invention is the activation agent. The activation agent has a number of important properties. For instance, the activation agent must be capable of being activated by an activation condition. The activation agent must also be capable of destroying or disrupting the cellular biochemistry of the cell or cell membrane it is or becomes incorporated into. Activation agents may be capable of being transferred or incorporated into the cell membranes or cellular interior of other. They may also have the capability of destroying or disrupting nearby or adjacent cells. The activation agent must also be capable of being on the surface of the encapsulation vesicles. Another important property of the activation agent is the fact that it is pre-assembled on the encapsulation vesicles before they are used in vitro or in vivo. There is no assembly process in vitro or in vivo and the activation agent is already in place on the surface of the encapsulation vesicles. In certain cases the activation agents may be self-assembling or have lytic activity. The activation agents may for instance comprise a zeolite, a nanotube, a nanorod, a nanocomposite, a nanowire, a nanodot, a quantum dot, a nanostructure, a plastic, a polymer, a synthetic material, silica or silicon materials, artificial bone or bone material, suicide inhibitors and other similar materials known and previously described in the art. The activation agents may also comprise a pore forming agent. The invention should not be interpreted to be limited to the above described embodiments and materials and includes other embodiments and materials that maintain the above described properties that are know in the art or that may be developed.

Pore Forming Agents:

One type of activation agent may be a pore forming agent. The pore forming agents have a number of important properties. The pore forming agent may have lytic activity. The pore forming agent must also be capable of activation by an activation condition. The pore forming agent may be capable of being activated to open, close or both. It also may be capable of releasing chemicals or molecules that may prove toxic to a pathogenic cell. Pore forming agents must be capable of being on the surface of an encapsulation vesicle. In certain instance, the pore forming agent is self assembling. However, self assembly is not a requirement of the agent. For instance, the pore forming agents of the present invention may comprise a biomolecule or solid-state material. Biomolecules may also include fusion proteins that may be a pore forming agent or may be used in conjunction with a pore forming agent to dock with a cell membrane or receptor on a cell membrane or surface. Pore forming agents may be designed to hold bioactive agents or degrade to release bioactive agents or other materials that may be potentially toxic to a pathogenic cell upon activation by an activation condition.

Biomolecules

In one embodiment of the invention a lytic pore forming agent may be used that is naturally occurring or synthetically made. The pore forming agent can be a molecule or fragment, derivative or analog of such molecules. The pore forming agents may be capable of making one or more lesions or pores in the encapsulation vesicle(s). These pore forming agents may be derived from a variety of bacteria including α-hemolysin, E.coli hemolysin, E.coli colicin, B. thuringensis toxin, aerolysin, perfringolysin, pneumolysin, streptolysin O, and listeriolysin. Eucaryotic pore forming agents capable of lysing cells include defensin, magainin, complement, gramicidin, mellitin, perforin, yeast killer toxin and histolysin. Synthetic organic molecules that are capable of forming a lytic pore in encapsulation vesicles can also be used. Other synthetic pore forming agents described in Regen et al, Biochem. Biophys. Res. Commun. 159:566-571, 1989, herein incorporate by reference.

The composition of the invention can also include fragments of naturally occurring or synthetic pore forming agents that exhibit lytic activity. In addition, the invention provides for biologically active and inactive fragments of polypeptides. Biologically active fragments are active if they are capable of forming one or more lesions or pores in synthetic or naturally occurring membrane systems. Inactive fragments are pore forming agents that are capable of being activated or cleaved into activity by some internal or external event, physical activity, or chemical modification.

The biologically active fragments of lytic pore forming agents can be generated by methods know to those skilled in the art such as proteolytic cleavage or recombinant plasmids.

The invention also includes analogs of naturally occurring pore forming agents that may be capable of lysing cells. These analogs may differ from the naturally occurring pore forming agents by amino acid sequence differences or by modifications which do not affect sequence, or both.

Modifications include in vivo or in vitro chemical derivatization of polypeptides, e.g., acetylation, or carboxylation. Also included in the spirit of the invention are modifications of glycosylation and those made by modifying the glycosylation patterns of a polypeptide during its synthesis and processing steps.

The invention also includes analogs in which one or more peptide bonds have been removed and replaced with an alternative type of bond or an alternative type of covalent bond such as a “peptide mimetic”. These mimetics are well known in the art. Similarly, the replacement of the L-amino acid residues is a standard way of rendering the polypeptide less sensitive to proteolysis. Also included are blocking groups that are used at the amino terminal end including: t-butyloxycarbonyl, acetyl, theyl, succinyl, methoxysuccinyl, suberyl, adipyl, azelayl, dansyl, benzyloxcarbonyl, fluorenylmethoxycarbonyl, methoxyazelayl, methoxyadipyl, methoxysuberyl, and 2,4 dinitrophenyl.

Although most modifications are designed to make most proteins more resistant to degradation, the present invention also includes modifications that may be used to enhance such modifications or degradations.

Also within the scope of the present invention are naturally or synthetically occurring organic and inorganic molecules that may be combined with proteins or constructs of the present invention to make them less susceptible to immunological attack. For instance, the compound of the present invention may be coupled to molecules such as polyethylene glycol (PEG) or monomethoxy-polyethylene glycol (mPEG).

The invention also includes modifications that result in an inactive pore forming agent that can be activated by cell associated substances or conditions. Such modifications can include peptides containing enzymatic cleavage sites (lysine and arginine bonds that can be cleaved) or chemically reactive groups that can be photo-activated. Modifications also include peptides that may be modified to optimize solubility properties or to mediate activation by cell-associated substances.

The invention also includes peptides and genetic variants both natural and induced. Induced mutants can be made in a variety of methods known in the art including random mutangenesis or polymerase chain reaction.

Solid-State Materials

The pore forming agents may also comprise solid-state materials that are capable of being on the surface of an encapsulation vesicle. For instance, the pore forming agent may be or may comprise a zeolite, a nanotube, a nanorod, a nanocomposite, a nanowire, a nanodot, a quantum dot, a nanostructure, a plastic, a synthetic material, silica or silicon materials, artificial bone or bone material, suicide inhibitors and other similar materials known and previously described in the art. Each of these materials must be capable of activation by an activation condition. Activation may include lytic activity and/or degradation or release of materials that may prove toxic to a pathogenic cell. The pore forming agent may also comprise a combination or mixture of one or more of these agents.

Targeting Ligand:

Targeting ligand refers to any material or substance that may promote targeting of tissues and/or receptors in vivo or in vitro with the compositions of the present invention. The targeting ligand may be optional employed with the present invention. A key property of the targeting ligand is the ability for the ligand to bind, attach or associate with the surface of a pathogenic cell. The targeting ligand provides the ability to distinguish between healthy and pathogenic cells.

The targeting ligand may be synthetic, semi-synthetic, or naturally occurring. Materials or substances that may serve as targeting ligands include, for example, proteins, antibodies, antibody fragments, hormones, hormone analogues, glycoproteins and lectins, peptides, polypeptides, amino acids, sugars, saccharides, including monosaccharides and polysaccharides, carbohydrates, vitamins, steriods, steriod analogs, hormones, cofactors, bioactive agents, genetic material, including nucleotides, nucleosides, nucleotide acid constructs and polynucleotides. The targeting ligands may include fusion proteins, monoclonal or polyclonal antibodies, Fv fragments, Fab′ or (Fab′)₂ or any similar reactive immunolgically derived component that may be used for targeting the constructs. Targeting ligands can also include other ligands, hormones, growth hormones, opiod peptides, insulin, epidermal growth factor, insulin like growth factor, tumor necrosis factors, cytokines, fibroblasts or fibroblast growth factors, interleukins, melanocyte stimulating hormone, receptors, viruses, cancer cells, immune cells, B cells, T-cells, CD4 or CD4 soluble fragments, lectins, concavalins, glycoproteins, molecules of hemopoetic origin, integrins and adhesion molecules. Other targeting ligands may be used in conjunction with the photodynamic pore forming agents. For instance, the seringe portion of the diphtheria toxin may be attached to a ligand and the constructs inserted into the encapsulation vesicles. These constructs could then be used to target or deliver the vesicles with the photodynamic pore forming agents.

Linkage of Targeting Ligands to Activation Agents and/or Encapsulation Vesicles:

The optional targeting ligands may be linked to either or both the activation agents and the encapsulation vesicles by means of covalent or non-covalent bonds. Non-covalent interactions include, but are not limited to ionic, dipole-dipole, van der waals, hydrophobic, hydrophilic, leucine-zipper or antibody-Protein G interactions.

A number of covalent linkages are possible. However, the preferred method when using the pore forming agents is that DNA encoding the proteins may be modified to include a unique cysteine codon. The second component that is used to bond with the cysteine can be derivatized with a sulfhydryl group. Cysteines and sulfhydryl groups can be introduced in a variety of methods including solid phase synthesis methods that are well known in the art.

Proteins can be modified by using standard techniques in the literature including agents such as Traut's reagent (2-iminothiolane-HCL) for primary amines, lysine residues, or N-terminal amines. A protein or membrane protein that has been modified with Traut's reagent can then react with proteins or peptides that have been modified with N-succinimdyl 3-(2-pyridyldithio)propionate (SPDP) or succinimidyl 4-(N-maleimidomethyl)cyclohexane-1-carboxylate (SMCC).

The free sulfhydryl groups can be generated by any known techniques in the art. In particular papain or pepsin may be used with antibodies. Pepsin being the preferred enzyme that cleaves on the carboxyl terminal side of disulfide bonds to produce (Fab′)₂ type fragments. These fragments can then be gently reduced using dithiothreitol or 2-mercaptoethanol to yield free sulfhydryl groups containing Fab′ fragments. Cysteines can also be produced in Fv or fusion proteins by means of recombinantly or genetically engineered methods known in the art.

Once the correct sulfhydryl groups are present on the appropriate fragments, encapsulation vesicles and/or targeting components, the sulfur groups of each component can be reduced, the compounds mixed and disulfide bond formation allowed to proceed at room temperature. Other organic or synthetic methods may be used that are well known in the art.

It is also within the spirit of the invention, that the targeting ligands may be directly attached to the encapsulation vesicle or to a different protein, molecule or biological component embedded in the encapsulation vesicle.

It should be understood that numerous groups could be used in actually coupling a bioactive agent to an encapsulation vesicle. Coupling can be achieved by using an active agent that has a reactive group that binds to the vesicle and a second reactive group that binds to the targeting agent, antibody or pore forming protein. Numerous methods exist in the art for these types of coupling reactions. These methods include and are not limited to cross-linking agents such as bifunctional reagents of homobifunctional or heterobifunctional type are well known in the art.

It should also be understood that there are numerous groups that can be used to couple pore forming or bioactive agents directly to the encapsulation vesicles. These groups comprise and are not limited to: NH_{2,} COOH, SH and OH groups that are found in abundance in the constituents of the encapsulation vesicles. A variety of important molecules and ligands including antibodies and polyethyleneglycol (PEG) or chemical modifiers such as iminothiolane can be used to conjugate to the membrane surface of the microvesicles or nanoerythrosomes. For instance, M344, α-HL, or its mutants (K8A, S3C, H35C, R104C) or the 48-127 antibody could be attached to the nanoerythrosomes directly by using —NH₂ groups of lysine or arginine residues or the —SH group of cysteine residues on the nanoerythrosome membranes. Linking arms have proven particularly useful in conjugating PEGs or other similar molecules to these membranes. Techniques using heterobifutinctional linking arms such as SMCC with the formula:

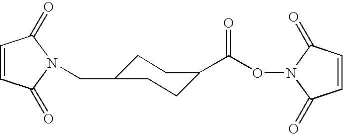

or a heterobifunctional linking arms of the following formula:

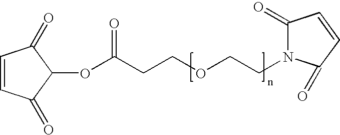

, wherein n=2 to 100.

Further modifications of proteins present in the membrane may be accomplished by using iminothiolane to add SH groups to the membrane or by conjugating the PEG or antibodies using electrophilic groups such as maleimides (Ex: SMCC and the heterobifunctional PEGs). COOH groups or anionic charges can be placed on the encapsulation vesicle by using anhydrides such as succinic, cis aconitic, and citraconic. In contrast, positive charges can be added to the encapsulation vesicle by using polyamines, amines, and amine/amino derivatives that will add —NH₂ groups to the surface of the encapsulation vesicle. Numerous type examples are prevalent in the art and include, but are not limited to putrecine, spermidine, and polylysine derivatives. These positive and negative charged membranes are useful for providing additional means for diagnostic separation of bound from unbound encapsulation vesicles.

A number of important frameworks exist for the coupling of PEG to encapsulation vesicles to improve the overall imunogenic properties of these vesicles. The following formula has been used as a means for describing the components that are effective for the formation of Vesicle-PEG conjugates:

Z-(YQ)-X—(CH₂—CH₂—O)ₘ—(CH₂)ₙ—W—R

The “Z” group is responsible for attaching the PEGs to proteins present in the nEryt membranes. This non-exposed group is very important for the overall kinetics of PEG conjugation to the available membrane reactive groups. The reactive groups that the “Z” group could bind or interact with include and are not limited to amino groups such as lysine residues. Two types of reactive groups targeting two families of nucleophiles present on proteins are particularly useful. These groups include NH₂ groups (i.e. lysine residues) exemplified by activated esters such as succinimide esters and SH groups (i.e. cysteine residues or iminothiolane derivatives of lysine), exemplified by maleimides, 2-thipyridyl derivatives and disulfide groups.

In its preferred embodiment, “Z” is selected from the group consisting of: COOH, HO, H (aldehyde), OH (hydroxyl) NH₂ and SH when YQ=(CH═CH) or —C(R″)═CH₂. In addition, if Y═—C═(O)—, then Z=H, N₃, OH, CH₃, —NH—NH_{2,} an anhydride, mixed anhydride, combinations or an activated ester. Further examples are known to those in that art and are further described in M. Bodansky in Principles of peptide synthesis: chapter II, Activation and coupling (Haftner et al., 1984, eds, Springer-Verlag, New York, pp 9-52).

Numerous methods exist for activating groups for binding PEG to encapsulation vesicles and include: cyanogen bromide (BrCN), amino acid ester (Zalipsky et al., 1984, J. Macromol. Sci. Chem. A21: 8339; Mutter et al., 1979, The Peptides; Gross et al., J., eds. 2, p. 285, Academic Press, New York), hydrazine and derivates, Rubinstein, 1978, U.S. Pat. No. 4,101,380; Davis et al., 1979, U.S. Pat. No. 4,179,337 and Persson et al., 1988, J. Chromatog. 457: 183′ succinimidyl carbonate derivates, Miron et al., 1993, Bioconjugate Chem. 4: 568; Zalipsky et al., 1993, Bioconjugate chem. 4: 296 and Zalipsky et al, 1991, Polymeric Drugs and Drug Delivery Systems; Dunn et al., eds., ACS, Washington, D.C.); oxycarbonlyimidazole derivates, Allen et al., 1991, Biochem. Biophys. Acta 1066: 29; and Tondelli et al., 1985, J. Controlled Release 1:25); Nitrophenyl carbonate derivatives, Satore et al., 1991, Appl. Biochem. Biotech. 27:45: tresylate derivatives, Klibanov et al., 1991, Biochem. Biophys. Acta 1062: 142, Delgado et al., 1990, Biotech. Appl. Biochem. 12:119); maleimide derivates, Kogan, 1992, Synthetic Commun. 22:2417 and Romani et al., 1984, Chemistry of Peptides and Proteins, Volter et al., eds, 2, p. 29, Walter de Gruyter, Berlin).

In order to attach the PEG to the proteins in the encapsulation vesicles, a “YQ” linking arm must also be used. This group is a very critical group since there are limited ways for attaching the PEG molecules to proteins. In its preferred embodiment, YQ is preferably composed of (CH₂)ₙ, where n=1 to 8 carbons atoms. Greater than 8 carbon atoms tends to render the complex to soluble in the membranes. In addition, it is very difficult to conjugate OH groups of PEGs to proteins. One exception is the cyanuryl chloride derivatives that have proven quite useful in preliminary experiments with encapsulation vesicles. YQ is a member selected from the group consisting of: CH═CH, —C(R″)CH₂ (where R′=lower alkyl of 1 to 5 carbon atoms), cyanuric chloride, cynaogen halides (Br or Cl) and other OH activating agents such as tosyl and mesyl groups. If Y is a lower alkyl [(CH₂)ₙ; n=1-7)]; then Q=—C(O), N, and S.

The “X” atom that connects the PEG molecule to the Z-(YQ) portion of the molecule is selected from the group consisting of: sulfur (thioether), oxygen (ether), —N—C(O) (amide), —S—C(O)(thioester), —O—C(O)(ester).

The (CH₂—CH₂—O)ₘ moiety of the molecule is the PEG itself and m could be anything from between 1 and 500. PEGs with molecular weights of 350 to 10,000 may be used. Preferably the PEGs will be in the range of 2,000-5,000 molecular weight range.

Lastly, the [(CH₂)ₙ—W—R] group that faces the cytosol or media surrounding the encapsulation vesicle must be considered. This group is particularly important to the present invention.

For example, (CH₂)ₙ—W—R should be an inert group such as OCH₃ (WR) in order to avoid immune responses in the host or in therapeutic applications. However, in other applications the [(CH₂)ₙ—W—R] group may be electropositively (—NH₂) or electronegatively (COOH) charged. Most applicable to the present invention, [(CH₂)ₙ—W—R] group could be SH, or 2-thiopyridyl or maleimide that could be used for either attaching the 48-127 antibody or conjugating all or part of the pore forming agents to the encapsulation vesicles. In particular, α-HL with or without attached antibody could be conjugated to the encapsulation vesicles by means of this group. Also as will be known to those in the art a variety of α-HL mutants or “pro-immunolysins” could be used or applied to the [(CH₂)ₙ—W—R] group. These proteins could be in an active or inactive form that is capable of being triggered into action under a defined chemical, physical or biochemical condition.

Preferably, in the formula [(CH₂)ₙ—W—R], n is =1 to 7 carbon atoms. In addition, W is selected from the group consisting of: O, N, S, and —C(═O). In addition, if W═—O—, R is a member selected from the group consisting of: lower alkyl, H, or cyloalkyl of 1 to 7 atoms or —C(═O)—R, where R is a polyamine, polyamine derivative, spermine, spermine derivatives or putrecine.

If W═—N—, the R is a member selected from the group consisting of: H, lower alkyl, or cycloalkyl of 1 to 7 carbon atoms, —C(═O)— or —C(═O)—R₂ where R₂ is selected from the group consisting of: spermine, spermidine, putrecine, or a lower alkyl chain of 1 to 6 carbon atoms having one or more PO₄, SO₃H or COOH group or groups.

If W═—S—, R is a member selected from the group consisting of: lower alkyl, cycloalkyl of 1 to 7 carbon atoms, —C(═O)—, —C(═O)—R and H, where R is a polyamine deriving from spermine, putrecine, or spermidine.

If W═—C(═O)—, R is a member selected from the group consisting of: lower alkyl, cycloalkyl of 1 to 7 carbon atoms,—C(═O)—, —C(═O)—R and H, where R is a polyamine deriving from spermine, putrecine, or spermidine.

WR is a member selected from the group consisting of: COOH, PO₄, and SO₃H. In addition, it should be mentioned the correct amount of substitution of PEG or other ligands is important to maintain the integrity of the encapsulation vesicles. If substitution is too high it is likely to cause the encapsulation vesicles to collapse. Preferably, 2-30% of the reactive groups of the membrane of the encapsulation vesicles will be substituted with PEG, more preferably 2-15%.

The Encapsulation Vesicle:

The encapsulation vesicle is important to the present invention and has a few important properties. The encapsulation vesicle must be capable of accomodating an activation agent on its surface. This may include the option of being able to attach a targeting ligand to the surface of the encapsulation vesicle. The encapsulation vesicle may also have the ability to encapsulate a bioactive compound. In addition, the encapsulation vesicle need not be a synthesized material. For instance, it may be naturally occurring or comprise parts of naturally occurring cells. For instance, the encapsulation vesicle may comprise a red blood cell, white blood cell, red blood cell ghost, white blood cell ghost, pathogenic cell, diseased cell, or any other cell that has been infected or not infected. However, as discussed above, the encapsulation vesicle must be capable of associating one or more activation agents on its surface. In other cases the encapsulation vesicles may be synthetic.

For instance in certain instances “vesicle” or “encapsulation vesicle” refers to an entity that is generally characterized by the presence of one or more walls or membranes that form one or more internal voids. Vesicles may be formulated, for example, from a stabilizing material such as a lipid, including the various lipids described herein, a proteinaceous material, including the various proteins described herein, and a polymeric material, including the various polymeric materials described herein. As discussed herein, vesicles may also be formulated from carbohydrates, surfactants, and other stabilizing materials, as desired. The lipids, proteins, polymers and/or other vesicle forming stabilizing materials, may be natural, synthetic or semi-synthetic. Preferred vesicles are those which comprise walls or membranes formulated from lipids. The walls or membranes may be concentric or otherwise. The stabilizing compounds may be in the form of one or more monolayers or bilayers. In the case of more than one monolayer or bilayer, the monolayers or bilayers may be concentric. Stabilizing compounds may be used to form a unilamellar vesicle (comprised of one monolayer or bilayer), an oligolamellar vesicle (comprised of more than about three monolayers or bilayers). The walls or membranes of vesicles may be substantially solid (uniform), or referred to as, for example, liposomes, lipospheres, stealth liposomes, nanoliposomes, particles, nanoparticles, micelles, bubbles, microbubbles, microspheres, nanospheres, nanostructures, microballoons, microcapsules, aerogels, clathrate bound vesicles, hexagonal/cubic/hexagonal II phase structures, and the like. The internal void of the vesicle may be filled with a wide variety of materials including, for example, water, oil, gases, gaseous precursors, liquids, fluorinated compounds or liquids, liquid perfluorocarbons, liquid perfluoroethers, therapeutics, bioactive agents, if desired, and/or other materials. The vesicles may also comprise a targeting ligand if desired. The encapsulation vesicles may also include nanoerythrosomes and other lipid based or cellular derived materials. In addition, the vesicles may comprise parts of cell, other diseased or pathogenic cells capable of fusion or having receptors or fusion proteins on their surfaces. For instance, a potential encapsulation vesicle may comprise a virus such as a T4 phage, an adenovirus, a polio virus, an influenza virus, an HIV virus or other viruses, bacteria, fungi, or pathogenic cells capable of membrane fusion. These vesicles may be naturally occurring or may have been altered physically or chemically through recombinant DNA technology. However, other naturally occurring or non-naturally occurring synthetic and non-synthetic organic or biologically based molecules, polymers and co-polymers are within the scope of the invention. Naturally occurring encapsulation vesicles may include erythrocytes, leukocyte, melanocytes, fibroblasts or components of these cells. Other encapsulation vesicles may include synthetically designed organic molecules and biodegradable polymers are also within the scope of the present invention.

In other embodiments of the invention, the vesicle may comprise a solid, substantially solid, gel, sol-gel, composite, nanocomposite, nanostructure, nanoporous material, porous nanostructure, degradable polymer, biodegradable polymer, or device as taught in U.S. Pat. No. 3,948,254 (herein incorporated by reference). Other structures well known in may include nanostructures that self-assemble. For instance such structures are described by Whitesides et al., Science (1991) 254: 1312-1319. Bates, Science (1991) 251: 898-905; Gunther & Stupp, Langmuir (2001) 17:6530-6539; Hulteen et al., J. Am. Chem. Soc. (1998) 120: 6603-6604; Moore and Stupp., J. Am. Chem. Soc. (1992): 9-14; Muthukumar et al, Science (1997) 277:1225-1232; Stupp et al., Science (1997) 276:384-389; Stupp et al., Science (1993) 259:59-63; and Zubarev et al., Science (1999) 283:523-526.

Activation Conditions or Substances:

An important component of the invention is the activation conditions that activate the activation agents. For instance, an activation agent such as a pore forming agent may be activated at the surface of the target cell by conditions or substances that are endogenously provided by the system or target cell or exogenously provided by a source other than the target cell. Physical, chemical or biochemical conditions may be used to activate the lytic activity. Physical conditions include heat, light or temperature changes. Chemical activators include changes in pH or reduction potential, metal ions or protecting groups that may be activated or de-activated. Light sources may include lasers, red lasers, ultraviolet lights, and other optical materials or substances well known in the art. Light wavelengths may include and not be limited to 400 nm, 500-550 nm, 630-650 nm etc.

In one embodiment of the invention, a removable photoactivatable protecting group may be employed. The pore forming agent or protein becomes inactive by addition of the protecting group. Upon irradiation by an external light or UV source the protecting group is removed and the pore forming agent becomes activated to form pores. Lytic pore forming activity can also be activated biochemically by any substance secreted by a pathogenic cell. These biochemical activators include: proteases, esterases, glycosidase, ectokinases, phosphatases and similar type substance or parts of these substances.

Assembly of the System:

The composition of the present invention or components such as the activation agents or pore forming agents may be self-assembling. The composition may be assembled in any order. The composition may be self-assembling or may be assembled manually in a step-wise fashion. It is important to the invention that the composition or activation agents be assembled on the surface of the encapsulation vesicles before they are used in vivo or in vitro. This insures that they will then be capable of activation by the activation conditions. Self-assembly may be molecular based where there is a spontaneous association of molecules under equilibrium conditions that form stable, structurally well defined aggregates joined by covalent or non-covalent bonds. Covalent, ionic, dipole-dipole or noncovalent bonds may also be used to attach pore forming agents to encapsulation vesicles. Pore forming agents may be positioned on the exterior or be embedded on the encapsulation vesicles. The invention also includes pore forming agents such as biomolecules that may be in monomeric or oligomeric forms embedded in the encapsulation vesicles. Components need not be spatially close together, but may be triggered to self-assemble upon an endogenous or exogenous condition, chemical or biochemical reaction or response.

Therapeutic Administration:

The composition can be administered to an animal or human suffering from a medical disorder or disease. The composition may be used alone or in combination with other chemotherapeutic or cytotoxic agents. The encapsulation vesicles can contain a bioactive agent used to treat a disease. For example an oligomeric antisense DNA or therapeutic charge could be used in the carrier for delivery to a diseased or pathogenic cell. Other bioactive agents used for treating cancer and HIV could also be used.

The composition may also be administered by intravenous infusion, subcutaneous injection, or direct injection to the site of infection. The present invention could also be applied topically or aspirated to a tumor site via bronchial passages to treat cancers of the lung. The therapeutic has the unique ability to operate at the cell membrane surface similar to how the immune system operates to destroy diseased cells. The present invention improves over drugs, prodrugs and bioactive agents since these agents over time may mutate, may become inactivated by diseased cells that are resistant to the drug or bioactive agent. In addition, the present invention is unique in that it teaches away from a number of new technologies and the trend of drugs being developed by pharmaceutical companies. For instance, one of the most anticipated of drugs entering approval is Fuzeon, commonly called T-20 that is being developed by Hoffmann La Roche and Trimeris Inc. Fuzeon is the first member of a new family of drugs called fusion inhibitors that are designed to prevent HIV from fusing with the membrane of target cells, the first step in viral entry. The present invention actually teaches away from this technology and the state of the art in that it may capitalize on the fusion process to increase destruction of pathogenic or diseased cells. Fuzeon is a peptide that must be administered through injection.

The present invention also capitalizes on other important interactions with cells. For instance, encapsulation vesicles may be taken up by a diseased cell by a number of mechanisms including contact release, adsorption, fusion, phagocytosis/endocytosis. In vivo, however, fusion often takes second place to phagocytosis. Under most circumstance, liposomes are cleared far to rapidly from the bloodstream by phagocytic cells for fusion events to occur to any significant event. However materials such as gangliosides, Sendai virus fusion proteins (active fusogenic reconstituted Sendai envelopes (RESVs)), lysolecithin, phosphatidyl ethanolamine, oleic acid, positively charged lipids, detergents and surfactants may be used to increase the rate of the fusion process. More favorable setting for fusion than the reticular endothelial system (RES) would include such areas as the aqueous humor of the eye, cerebrospinal fluid, or following passive absorption to the walls of capillaries (See New, R. R. C. Liposome: A Practical Approach, Oxford University Press, 1997: Chapter 2, 85-90; Chapter 6, 221-239). The present invention has the capability of working with all form of incorporation including but not limited to receptor mediated endocytosis, endocytosis, phagocytosis and pinocytosis. In this case, the pore forming agent can be photodynamically activated and will help speed up the release of the encapsulated contents into the endosomes.

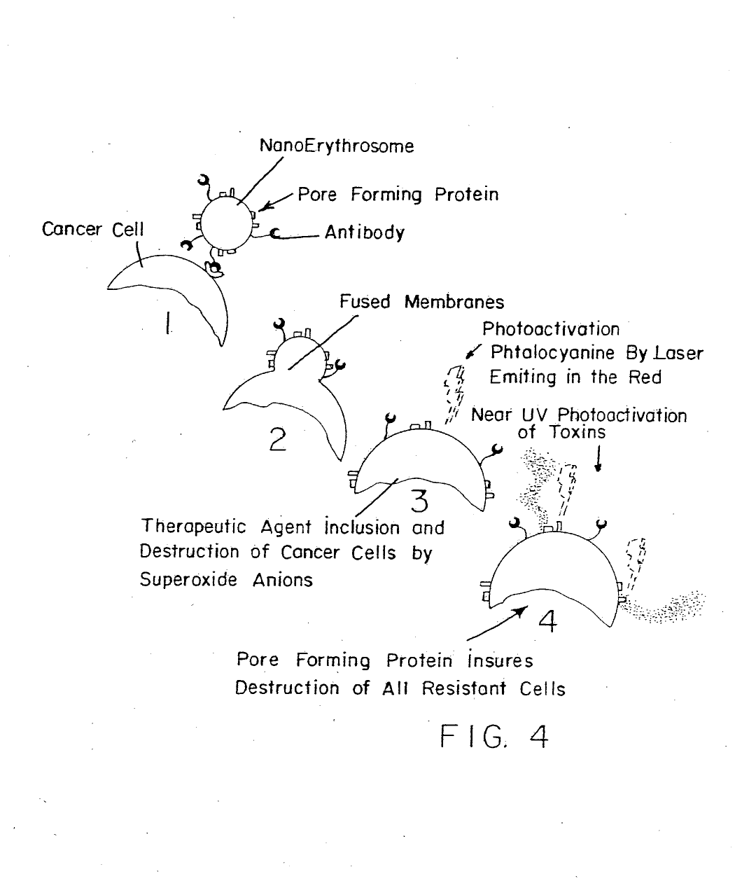

FIG. 4 shows a diagram of how the therapeutic may operate to destroy drug resistant cells. Although the figure shows a particular embodiment of the invention, the drawing is provided for illustrative purpose only. The scope of the present invention should not be construed to be limited to this particular embodiment. Other broad embodiments, applications and components of the invention are illustrated and provided throughout the disclosure.

An encapsulation vesicle such as for example a nanoerythrosome with embedded photo-activatable pore forming agent (1) and attached targeting ligands is used to recognize a specific tumor antigen. The construct is retained by the cancer cell and the therapeutic agent is transferred into the cell cytoplasm (2) via a fusion mechanism or absorption process. The therapeutic then interferes with the cancer cell's functions and destroys the cell (3 and 4). The inactivated pore forming agent with attached protecting group is incorporated into the cancer cell's membrane and may be photo-activated upon irradiating by an external light source. The photo-generated pore forming agents are used to guarantee destruction of any therapeutically resistant cancer cells.

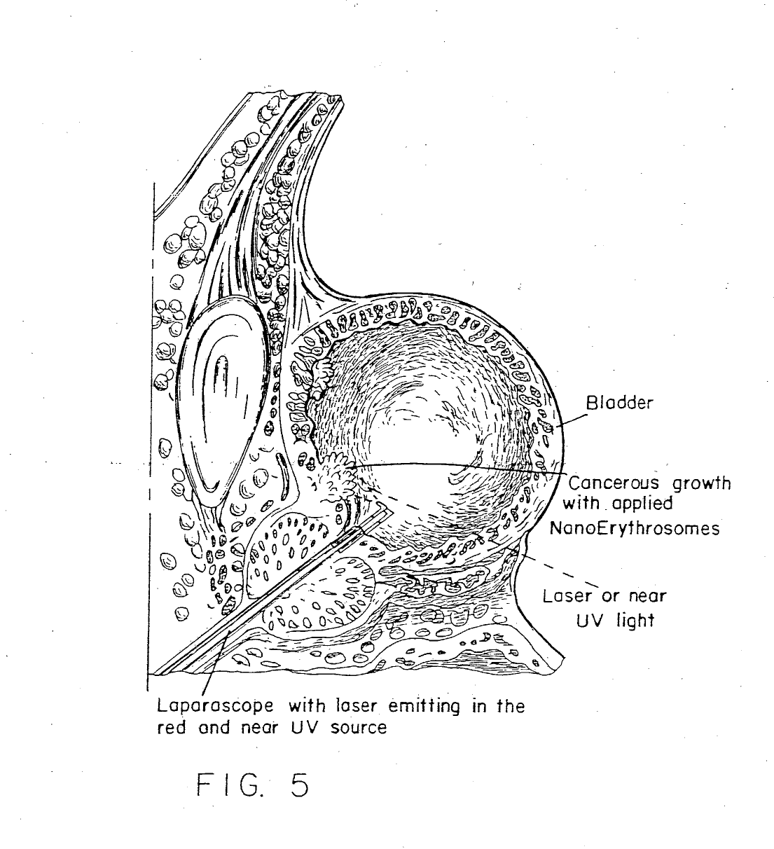

The present invention could be used to treat certain topical skin cancers or intravesical bladder cancers. For such a treatment, vesicles encapsulating a photosensitive phthalocyanine could be used. More specifically, specific antibodies conjugated to the vesicle target a particular cancer of the bladder or skin. The composition is manually introduced into the bladder of the patient or animal, and applied topically. Subsequently, a fiber optic device or laparascope, linked to a laser emitting in the red is introduced into the bladder to activate the phthalocyanine (See FIG. 5), thereby inducing important increases in the intracellular superoxide anions which provokes the death of the cells targeted by the antibody. In addition, the fiber optic device or laparascope will be designed to include a linked UV irradiation source for separately activating the pore forming agents upon command.

### EXAMPLE 1

### Preparation of an Encapsulation Vesicle

a. Preparation of Rabbit Nanoerythrosomes:

The nanoerythrosomes were prepared according to the procedure described below. Initial experiments were conducted with rabbit erythrocytes because of their high susceptibility to take-up α-hemolysin pore forming proteins.

1. Removal of Plasma and White blood Cells:

Rabbit blood was collected by cardiac puncture and the first few milliliters discarded. Blood (2×30 ml) was centrifuged at 500×g (1500 rpm) for 10 minutes at 4° C. The white blood cells and the plasma was then removed by aspiration. The volume that was removed was replaced by the same volume of phosphate buffer saline (PBS) (sodium phosphate 5 mM, sodium chloride 150 mM) at pH 7.4 and at 4° C. This mixture was then mixed and inverted several times and re-centrifuged at 500×g for 10 minutes at 4° C. The sequence was repeated 3 times. The remaining steps and procedures were conducted under sterile conditions.

2. Preparation of Erythrocyte Ghosts:

Five milliliters of blood was prepared and treated as described above and was added to 35 mL (in 50 mL Nalgene™ tubes) of hypotonic buffer (sodium phosphate 5 mM) at pH 7.4. This suspension was then centrifuged at 25,000×g for 20 minutes at 4° C.

Next, the supernatant was aspirated and discarded. The volume of buffer removed was again replaced by a similar amount of fresh hypotonic buffer. The suspension was then centrifuged again and the procedure repeated until the supernatant was slightly colored (3-4 times). The pellet was then suspended in 5 mL's of PBS at pH 7.4 at 4° C. The white ghost suspensions were then pooled, concentrated by centrifugation (20,000×g) for 20 minutes and kept at 4° C. until needed.

3. Preparation of nanoErythrosomes (nEryt): Although a few different methods exist for the production of the nEryt, the following procedure was used because of its advantage of providing faster production for large-scale production as well as ability to provide higher yields.

A hypotonic suspension (0.2 mg/ml) of white blood cell ghosts in hypotonic PB at pH 7.4 was immediately filtered under vacuum through a polycarbonate (nylon or polyethersulfone) filter having pores of approximately 0.45 um, immediately followed by a second filtration through a second (nylon or polyethersulfone) filter having pores of approximately 0.20 um. The procedure was carried out in a hypotonic buffer that was (di or trivalent cation free). The mean diameter of nEryt produced according to this procedure was about 100-200 nm.

4. Concentration of nEryt Suspension:

NEryt suspensions (350 mL at 0.2 mg of protein per ml) was produced above, and was concentrated using an Amicon™ concentrator (Model 8400 having a cut-off smaller than 500,000 such as a ZM 500 or YM 100) to a volume of 50 mL under nitrogen pressure of 10 psi and gentle stirring.

5. Entrapment of Therapeutic Charge by nEryt:

Suspensions of nEryt (1.5 ml, 1 to 2 mg/ml) were centrifuged (microfuge™) at 16,000×g for 10 min. at 4° C. The supernatant was then removed and discarded and the protein content of the pellet was determined by a commercially available kit. One mL of the therapeutic charge was then added to the pellet. The suspension was then homogenized by several inversions and kept at 4° C. for 10 minutes. Next, the suspension was frozen in liquid nitrogen for 2 minutes and unfrozen in a water bath at 25° C. It should be noted that the heat-shock treatment need not be at −180° C., as freezing at −70° C. or −20° C. was used. It should also be noted that when DNA or other oligonucleotides were used, they could be observed following incubation at room temperature.

6. Purification of nEryt Having Entrapped Therapeutic Charge:

After the above procedure, the nEryt were freed from the untrapped molecules by 3 to 5 cycles of dilution with 1 ml of cold 4° C. PBS (isotonic at pH 7.4), centrifugation at 16,000×g for 8 minutes, removal of the supernatant and dilution again with PBS. In the case of entrapment of DNA or an oligonucleotide, two hundred nanograms of nEryt were suspended in PBS and then washed with a TKN buffer to eliminate phosphates. Approximately, 300 ng of DNA was added to 300 ul of the TKN buffer and then homogenized. This suspension was then incubated at 4° C. for 10 minutes and frozen in liquid nitrogen for 2 minutes. The suspension is then incubated at 25° C. for 4 minutes and treated with TKN buffer. Excess DNA was removed from the suspension by adding Dnase to the reaction mixture (100 ul of 0.1 U/ul Dnase 1 solution) and 10 mM Mg²⁺. Conventional PCR was then used to confirm the entrapment of the DNA in the nEryt.

7. Preparation of Sample for Lyophilization and Storage:

Samples were prepared for lyophilization by adding 1 mL (1 mg of suspension with bioactive agent in hypotonic PB at pH 7.4 or isotonic PBS. Five hundred microliters of the following solution were then added (Sucrose (26.3% p/v (770 mM), polyvinylpyrolidone (mw=10,000 18.1% p/v; (18 mM), EDTA (ethylenediamine tetraacetic acid trisodium salt; 1 mM =Lyophilized solution) dissolved in either PBS or Pbat pH 7.4. These samples were stable for 7 cycles of freezing at −20° C. (12 hours) thawing at 25° C. (16 h) and finally heating at 40° C. for (18 hours).

The steps in lyophilization were straight forward and include a typical preparation of around 1.5 mL of the suspension of nErt as prepared above in 10 mL vials frozen for 2 min. in liquid nitrogen. The suspension was lyophilized for 6 to 12 hours. A sucrose solution (sucrose 25.5% p/v (745 mM) in water or PBS) at 37° C. for 1 hour was added to the lyophilized nEryt to maintain the functionality of the bioactive agent.

Samples were stable for at least 7 months if stored at 4° C.

### EXAMPLE 2

### Synthesis and Application of BNPA

The novel protecting group 2-bromo-2-(2-nitrophenyl) acetic acid (BNPA) was used to make all proteins photactivatable. This water-soluble reagent places an a-carboxy-2-nitrobenzyl (CNB) group on sulfhydryl groups. BNPA was produced in high yield by the bromination of 2-nitroacetyl chloride, followed by hydrolysis of the acyl chloride group. BNPA is highly water-soluble at pH values around neutral.

The synthesis of BNPA α-halogenation of acyl chlorides was carried out by methods known and described in the art, e.g. Harpp et al., J. Org. Chemn. 1975, 40:3420-27. To 2-(2-nitrophenyl)acetic acid (Aldrich, 5.0 g, 27.6 mmol), was added carbon tetrachloride (5 mL) and thionyl chloride (7.95 mL, 109 mmol). The final mixture was then stirred at 65° C. for 1.5 hours to form acyl chloride. Subsequently, N-bromosuccinimide (5.90 g. 33.1 mmol), CCl₄ (25 mL) and a catalytic amount (10-12 drops) of HBr in acetic acid were added to the flask. The final mixture was then heated at 70° C. for around 4.5 hours and then ice was added (25 g) to cool the mixture. The mixture was stirred vigorously for hour to hydrolyze the acyl chloride. The organic layer was then retained and the aqueous layer extracted with 3×25 mL CH₂Cl₂. Combined fractions were then dried by Na₂SO₄ and the solvent removed by evaporation. Brown, oil-like BNPA was obtained in high yield and was re-crystallized twice from CH₂Cl₂ to yield a pure solid. UV and elemental analysis were in agreement with the results found and described by Chang et. al., 1995, Chemistry and Biology 2: 391-400.

### EXAMPLE 3

### Design, Preparation and Integration of the Photogenerated Toxins into Nanoerythrosomes

Production of α-hemolysins Using In vitro Transcription and Translation (IVTT):

Photoactivatable pore forming agents were made by modifying α-HL with 2-bromo-2-(nitrophenyl)acetic acid (BNPA). These methods are known in the art. An active single-cysteine mutant of α-HL was then inactivated by reaction with BNPA. This reaction introduced an α-carboxy-2-nitrobenzyl (CNB) group which is capable of removal upon photolysis and restoring the pore forming capabilities of α-HL. These photactivatable proteins have the advantage of being capable of permeabilizing selected or defined cells.

In vitro synthesis of α-HL polypeptides was carried out in an E.coli S30 extract supplemented with T7 RNA polymerase and rifampicin. 1.0 μg of plasmid DNA was added to a 25 μL reaction. The following mutant α-HL genes were inserted into a plasmid similar to pT7Sf1A (Walker et al., J. Biol. Chem., 1992 267:10902-10909). K8A, S3C, H35C and R104C. As described and known in the art, R104C was derived from K8A and S3C and H35C were derived from unaltered genes. K8A has the advantage of not being susceptible to cleavage by adventitious proteases.

BNPA Modification of Single-Cysteine Mutants:

α-HL mutants (60 μL IVTT mix) were added to 10 mM Dithiothreitol (DTT) (30 μL in water) at room temperature. A 1.0 M Tris HCL buffer (60 μL) in water (90 μL) was used (pH 8.6). A 100 mM BNPA in 100 mM NaPᵢ, pH =8.5 (60 μL) was then added to the mixture and incubated at room temperature in the dark for 2 hours (S3C) and 9 hours (H35C, R104C). Excess BNPA and extraneous low mass sulfhydryl products were removed from desired products by repeated dilution with 100 mM Tris-HCl and concentration using an Amicon, Microcon 3 ultrafiltration device. The final concentration of DTT was adjusted to be around 1 mM and the BNPA adducts in the solution to be less than 20 μM. All modified proteins were then stored in appropriate sub-ambient conditions that are well known in the art.

The α-HL R104C (0.36 mg/mL) was dialyzed against 10 mM Tris-HCL, pH 8.5 containing 5 mM 2-Mercaptoethanol. Dialyzed R104C, 1.0 M Tris-HCL and 10 mM DTT were incubated for 10 minutes at room temperature before the addition of the BNPA solution. After approximately 3 hours, 1.0 DTT was added to the solution and excess reagents removed by gel filtration (Bio-Gel 2) methods that are well known in the art (Buffer contained 10 mM Tris/HCL, pH 8.5. and 50 mM NaCl). Fractions were then desalted using 100 mM Tris-HCL, pH 8.5 and 1.0 mM DTT by ultrafiltration as described above.

Photolysis of CNB-αHL:

Photolysis of CNB-αHL in 100 mM Tris-HCL (pH 6.0 or 8.5) with 1 mM DTT was carried out in a microplate reader by irradiating the samples for 30 minutes with a UV 300 illuminator with a 285 nm cut-off filter. The assays or samples used for unmasking the protected cysteine residues were treated with IASD and then analyzed by SDS-PAGE. For the pH 8.5 sample, irradiated αHL was diluted with 100 mM Tris-HCL and then reacted with 100 mM IASD in water for 2 hours at room temperature. For the pH 6.0 samples, irradiated αHL was diluted with 1.0 M Tris-HCL, pH 8.5 and treated in a similar fashion.

Hemolysis Assays:

The lytic activity of the αHL polypeptide was measured by lysis of rabbit erythrocytes in the presence of the polypeptides. Both irradiated and unirradiated CNB-αHL in 100 mM Tris-HCL, pH 8.5, containing 1 mM DTT were placed in microplate readers and diluted with appropriate volumes of (K-PBSA) (20 mM KP, pH 7.4, 150 mM NaCl and 1 mg/mL BSA). Washed rabbit erythrocytes were then added to 0.5% and the plate was incubated at 22° C. for 3 hours. Hemolysis kinetics were recorded and readings taken by methods known in the art.

SDS-PAGE Electrophoresis:

SDS-PAGE electrophoresis was carried out according to Laemmli (Laemmli, Nature, 1970, 227:680-85) in 40 cm long 12% polyacrylamide gels run at constant voltage (200 V for 40 hours). Gels were then fixed using a standard methanol/water/acetic acid solution (3:6:1), dried and then subject to autoradiography.

Modification and Photodeprotection of αHL-S3C and αHL-R104C:

Construction, modification and photodeprotection experiments were conducted on αHL-S3C and αHL-R104C by methods well known in the art (Chang et al., 1995, Chemistry and Biology Vol. 2, No. 6 and U.S. Pat. No. 5,777,078). Mutants were tested to assure that generated photoproducts did not cause lysis of rabbit erythrocytes. After activation, appreciable lysis was observed with the αHL-R104C mutants, but not with the αHL-S3C mutants. SDS PAGE results for the αHL-S3C and αHL-R104C mutants showed similar band patterns to those results obtained by (Chang et al., 1995, Chemistry and Biology Vol. 2, No. 6 and U.S. Pat. No. 5,777,078). αHL-S3C appeared to undergo a large reduction in electrophoretic mobility upon modification by IASD (Krishnasastry et al., FEBS Lett., 1994, 356:66-71). Reaction of radio labeled αHL-S3C with BNPA was found to produce a substantial gel shift upon prolonged irradiation. In accordance with previously reported results, complete modification of αHL-S3C was achieved with 20 mM BNPA at pH 8.5 after 1 hour a room temperature. Excess reagent was removed by ultrafiltration. Modified αHL-S3C was irradiated at pH 6.0 and pH 8.5. Results were similar to those obtained by (Chang et al., 1995, Chemistry and Biology Vol. 2, No. 6 and U.S. Pat. No. 5,777,078) in which far less αHL-S3C-CNB was converted to αHL-S3C at pH 8.5.

αHL-R104C is as active at WT-αXHL before modification. Upon modification by IASD, it gives and especially pronounced gel shift with negligible activity. Similar to results reported, after treatment with BNPA pore forming activity was lost. The modification and deprotection experiments with near UV irradiation was examined by SDS PAGE as described above for αHL-S3C. Reaction with BNPA produced the expected decrease in electrophoretic mobility with about 80% conversion to αHL-R104C-CNB. Reaction products were then treated with IASD and the photochemistry of the BNPA modified products examined. The BNPA-modified αHL-R104C was irradiated at 300 nm at pH 6.0 and pH 8.5. The polypeptide presumed to be αHL-R104C was shifted to reduced mobility after treatment with IASD, demonstrating the presence of free sulfhydryl groups and the generation of αHL-R104C from BNPA-modified αHL-R104C. Based upon the amount of reaction with IASD, the yield was around 50-60%.

Incorporation and Testing in nEryt(s):

αHL-S3C and αHL-R104C mutants were then tested for pore forming activity in nanoerythrosomes. Hemolysis assays were performed with nanoerythrosomes derived from rabbit erythrocytes. WT-αHL that was treated with BNPA was included as a control to determine whether UV irradiation decreased the activity of αHL. In a second experiment, αHL-H35C-CNB was included to demonstrate that photoproducts generated from components of the in vitro protein synthesis and chemical modification did not cause lysis of rabbit erythrocyte nanoerythrosomes.

As expect, the WT-αHL had strong lytic activity before irradiation, while the αHL-S3C and αHL-R104C mutants showed no activity. After irradiation at 300 nm, the WT-αHL retained its activity while the irradiated αHL-S3C remained inactive. The mutant αHL-R104C was activated by the irradiation and showed similar results to those obtained and described in (Chang et al., 1995, Chemistry and Biology Vol. 2, No. 6 and U.S. Pat. No. 5,777,078). As determined by quantitative microtiter assays, the extent of activation was around 15%.

Although the action and activity of the αHL-R104C mutants have been regarded as being too slow for use with rapid transmembrane fluxes of Ca²⁺, these mutants are quite suitable for use with drug delivery, and topical or intravesical cancer therapeutics that are not time-dependent. Other lytic agents and mutants may be used that can create larger pores such as aerolysin, perfringolysin, pneumolysin, streptolysin, O. lysteriolysin, Bacillus thurigensis toxin, E.coli-derived hemolysin, E. Coli derived colicin, defensin magainin, mellitin, complement, perforin, yeast killer toxin and histolysin. Each of these agents can be modified according to the present invention to make photoactivatable pore forming agents that can permeabilize cells. In addition, it is within the scope of the invention that other cysteine modified or modifiable mutants can be produced and incorporated into similar drug delivery constructs that may operate at either lower or higher pH conditions than those disclosed by the present invention.

The photoactivatable pore forming agents may also be designed to be switched on or off rather than being just switched on. One approach would be to combine the photochemical trigger with another type of trigger that is well known in the art. For instance, a trigger for pore-closure could be designed in conjunction with the photactivatable trigger, for closing the pores by divalent ions. For example, the metal sensitive mutant α-HL-H5m, can be modified with BNPA and then incorporated into nanoerythrosomes. As a result, the α-HL-H5m-CNB compound can be activated by light, but the pores also have the capability of being closed by addition of low concentrations of divalent ions.

Other approaches for modification include the use of photoisomerizable groups for the targeted chemical modification of a single cysteine. Azobisbenzenes, spiropyrans and related molecules have been used to produce switchable enzymes by random protein modification.

### EXAMPLE 4

### Design and Preparation of the Targeting Ligand

The 48-127 murine monoclonal antibodies used for targeting the constructs were produced in accordance with methods well known in the art. The following procedure was used:

1. Preparation of Monoclonal Antibodies:

a. Tissue Culture:

Cultured cells of T-24 human bladder cancer were obtained from the collection of J. Fogh (Sloan-Kettering Institute). Other human cell lines and epithelial cells were maintained and produced according to previous described methods (Carey et al., 1976; Ueda et al., 1979).

b. Serological Tests and Procedures:

Rosetting assays for the detection of cells surface antigens using rabbit anti-mouse Ig conjugated to human 0 erythrocytes were performed as described by (Dippold et al., 1980). Absorption tests and Enzyme linked immunosorbent assays (ELISAs) were performed as described (Caimcross et al., 1982; Carey et al., 1976; Lloyd et al., 1983).

c. Immunization:

Mice will were immunized three times at intervals of 3 to 4 weeks by intraperitoneal innoculation of T16 Cells from bladder cancer lines.

d. Derivation of Mouse Monoclonal Antibodies:

Mice were sacrificed 3-4 days after immunization and spleen cells fused with mouse myeloma cells. Clones were selected according to reactivity of ELISA on cultured cancer cell lines. After subcloning three or four times, hybridomas were injected into mice and sera from growing tumors used for serological and biochemical cell tests.

e. Immunoflourescence Assays:

Frozen sections of tissue were fixed for 5 minutes with 3.7% formaldehyde in phosphate-buffer saline. Tissue sections were then washed and incubated for 1 hour with undiluted hybridoma culture supernatants. Slides were then washed and incubated for 30 minutes with a (1 to 40 dilution) of flourescein conjugated goat anti-mouse Ig. Samples were then wet mounted in a 90% glycerol in phosphate-buffered saline.

f. Immunoprecipitation Procedures:

Antibodies were tested using detergent solubilized cell extracts labeled with [³H]glucosamine.

2. Attaching the Targeting Ligands to the Encapsulation Vesicles:

A free sulfhydryl group of the antibody can be generated by methods known in the art. For example, the antibody was cleaved enzymatically with pepsin to yield (Fab′)₂ fragments, which were gently reduced with dithiothreitol (DTT)or 2-mercaptoethanol to yield free sulfhydryl groups containing Fab′ fragments. Antibody fragments and, e.g., single chain Fv, can also be expressed recombinantly and genetically engineered to contain a terminal cysteine group using methods known in the art or chemically modified as described and outlined above.

Once the correct sulfhydryl groups were present in the 48-127 antibody and encapsulation vesicles surface(s), the two components were purified and then reduced. Reduced compounds were then mixed and disulfide bond formation was allowed to proceed at room temperature. To improve the efficiency of the coupling reaction, the cysteine residue of one of the components was activated prior to mixing by addition of 5,5′-dithiobis(2-nitrobenzoic acid) (DTNB) or 2,2′ dithiopyridine using methods well known in the art. After these reactions were complete, the mixture was dialyzed against phosphate saline buffer to remove unconjugated molecules. Sephadex chromatography was then carried out to separate the compounds of the invention from any of its constituent parts.

3. Assembly of the Final Therapeutic Composition:

The method of assembly is to attach the targeting ligands to the encapsulation vesicles and then incorporate the pore forming photoactivatable proteins into the encapsulation vesicles.

4. Heptamer Formation in nEryts and Assay for Ligand Specificity:

The IVTT experiments to produce αHL-R104C and αHL-S3C mutants described above were carried out in the presence of (³⁵S) methionine (1200 Ci/mmol) and the reaction was stopped by the addition of choramphenicol (100 uM) and unlabelled methionine (5 mM), which prevented the incorporation of the ³⁵S into the rabbit nanoerythrosomes. IVTT mix (5 uL), was incubated with 10% nanoerythrosomes (50 uL) for 60 minutes at 20 degrees Celsius in K-PBSA. The cells or membranes were recovered by centrifugation, dissolved in 30 uL 1× loading buffer (U.K. Laemmli, Nature 227:680, 1970), warmed at 45 degrees Celsius for 5 minutes and subjected to electrophoresis in a 12% SDS-polyacrylamide gel. Complex products that formed heptamers were then assayed against the Trop-2 antigen present on bladder cancer cells (to test binding and specificity) and the products again subjected to electrophoresis in a 12% SDS-polyacrylamide gel.

While it will be apparent that the illustrated embodiments of the invention herein disclosed are calculated adequately to fulfill the objects and advantages primarily stated, it is to be understood that the invention is susceptible to variation, modification, and change within the spirit and scope of the subjoined claims.

The invention having been thus described, what is claimed as new and desired to secure as Letters Patent is:

### Appendix

### References

1. Arnold, F. H., & Haymore, B. L. (1991) Science 252,1796-1797.

2. Bainbridge, L. J.; Slade, J., Eds.; Current Medicine; Edward Brothers, Inc.: Philadelphia, Pa., 1996; pp 111-115.

3. Bander, N. H. Monoclonal antibodies in diagnosis and treatment of bladder cancer. In Bladder Cancer: Future Directions for Treatment; A. Yagoda (ed.); John Wiley & Sons : New York, 1986; pp 75-79.

4. Bayley, H. (1994) Journal of Cellular Biochemistry 56, 177-182.

5. Bayley, H. (1995) Bioorganic Chemistry 23, 340-354.

6. Bayley, H. (1997) Sci Am. 277, 62-67.

7. Bayley, H.; Walker, B. J. Cell Targeted Lytic Pore forming Agents. U.S. Pat. No. 5,817,771, Oct. 6, 1998a.

8. Bayley, H.; Walker, B. J. Cell-Targeted Lytic Pore forming Agents. U.S. Pat. No. 5,824,776, Oct. 20, 1998b.

9. Bayley, H.; Walker, B. J.; Chang, C.; Niblack, B.; Panchal, R. Triggered Pore forming Agents. U.S. Pat. No. 5,777,078, Jul. 7, 1998c.

10. Bernheimer, A. W., Kim, K. S., Remsen, C. C., Antanavage, J., and Watson, S. W. (1972) Infection and Immunity 6, 639-641.

11. Bernheimer, A. W. & Schwartz, L. L. (1963) J. Gen. Microbiol. 30,455-468.

12. Bhakdi, S., & Tranum-Jensen, J. (1991) Microbiol. Rev. 55, 733-751.

13. Bhakdi S., Weller, U., Walev, I., Martin, E., Jonas, D., & Palmer, M. (1993) Med. Microbiol. Immunol. 182, 167-175.

14. Blomqvist, L. & Thelestam, M. (1988) Microb. Pathog. 4, 223-229.

15. Blood, C. H., & Zetter, B. R. (1990) Biochim.Biophys.Acta 1032, 89-118.

16. Boman, H. G. & Hultmark, D. (1987) Ann. Rev. Microbiol. 41, 103-126.

17. Boman, H. G., Wade, D., Boman, I. A., Wahlin, B., & Merryfield, R. B. (1989) FEBS lett. 259, 103-106.

18. Boulnois, G. J., Paton, J. C., Mitchell, T. J., and Andrew, P. W. (1991) Mol. Microbiol. 5, 2611-2616.

19. Buckley, T., (1992) FEBS307, number 1, 30-33.

20. Cairncross, J. G., Mattes, M. J., Beresford, H. R., Albino, A. P., Houghton, A. N., Lloyd, K. O. & Old, L. J. (1982) Proc. Natl. Acad. Sci. USA 79, 5641-5645.

21. Cairns, J. (1989) Cancer Cells 1, 1-8.

22. Cancer Statistics Review 1973-1989; NIH Publication No. 92-2789; U.S. Department of Health and Human Services; National Cancer Institute: Bethesda, Md., 1992; XXVI.1.

23. Carey, T. E., Takahashi, T., Resnick, L. A., Oettgen, H. F. & Old, L. J. (1976) Proc. Natl. Acad. Sci. USA 73, 3278-3282.

24. Chang, C., Niblack, B., Walker, B. & Bayley, H. (1995) Chem. And Biol. 2, 391-400.

25. Chaudhary (1990) Acad. Sci. USA 87, 1066-1070.

26. Cooper, L. Z., Madoff, M. A., and Weinstein, L. (1966) J. Bacteriol. 91, 1686-1692.

27. Cowan, S. W., et al., & Rosenbusch (1992) Nature 358, 727-733.

28. Davies, A. J. S., & Crumpton, M. J. (1982) Cancer Surveys 1, 347-559.

29. de Harven, E., Fradet, Y., Connolly, J. G., Hanna, W., He, S., Wang, Y., Choi, B. C. K., McGroarty, R., Bootsma, G., Tilups, A., & Christensen, H. (1992) Cancer Res. 52, 3131-3137.

30. Dippold, W. G., Lloyd, K. O., Li, L. T. C., Ikeda, H., Oettgen, H. F. & Old, L. J. (1980) Proc. Natl. Acad. Sci. USA 77, 6114-6118.

31. Domellöf, L. In Drug Delivery in Cancer Treatment; Domellöf, L., Ed., Springer-Verlag Berlin Heidelberg: Germany, 1987; pp 86-89.

32. Embleton, M. J. (1987) Br J. Cancer 55, 227-231.

33. Erdmann, J. Gen. Eng. News 1999,19 (May 15) I.

34. Fradet, Y., Cordon-Cardo, C., Thomson, T., Daly M. E., Whitmore, W. F., Lloyd, K. O., Melamed, M. R., & Old, L. J. (1984) Proc. Natl. Acad. Sci. USA 81, 224-228.

35. Fradet, Y., Cordon-Cardo, C., Whitmore, W. F., Jr., Melamed, M. R. & Old, L. J. (1986) Cancer Res. 46, 5183-5188.

36. Füssle, R., Bhakdi, S., Sziegoleit, A., Tranum-Jensen, J., Kranz, T., and Wellensiek, H.-J. (1981) J.Cell Biol. 91, 83-94.

37. Gaudreault, R. C.; Gicquaud, C.; Poyet, P. Nanoerythrosome as Bioactive Agent Carrier. U.S. Pat. No. 5,653,999, Aug. 5, 1997.

38. Gaudreault, R. C. Polyethyleneglycol Conjugated Nanoerythrosomes, Method of Making Same and Use Thereof. WO 98/11919, Mar. 26, 1998a.

39. Gaudreault, R. C. Method of Capture of a Bioactive Agent by Nanoerythrosome Preparation. WO 98/11920, Mar. 26, 1998b.

40. Gauaux, J. E., et al., & Bayley, H. (1994) Proc. Natl Acad. Sci. USA 91, 12828-12831.

41. Glazer, R. I. Mechanisms of Drug Resistance. In Developments in Cancer Therapy; Glazer, R. I., Ed.; CRC Press, Inc.: Boca Raton, Fla., 1989; pp 119-120.

42. Gray, G. S., and Kehoe, M. (1984) Infect. Immun. 46, 615-618.

43. Herr, H. W., Laudone, V. P. & Whitmore, W. F., Jr. (1987) J. Urol. 138, 1363-1368.

44. Hopkins, C. R.: Site—Specific Drug Delivery—cellular opportunities and challenges. In: Site-Specific Drug Delivery. Cell Biology, Medical and Pharmaceutical Aspects; Tomlinson E. and Davis SS (eds) John Wiley, Chichester, 1986; pp 26-48.

45. Ihler, M., In: Drug Carriers in Biology and Medicine, Gregoriadis G. ed;. Academic Press: London, 1979; pp. 153-192.

46. Ihler, M., In: Methods of Drug Delivery, Ihler Ed.; Pergamon Press: Oxford, 1986; pp 3-21.

47. Jain, R. (1994) Sci. Am. 271, 58-65.

48. Janeway, C., In: Imimunobiology—The immune system in health and disease, Fourth Edition: Elsevier Science Ltd/Garland Publishing: London, 1999: pp 82-83.

49. Jursch, R., et al., & Bhakdi, S. (1994) Infect. Immun. 62, 2249-2256.

50. Kirkwood, J. M; Lotze, M. T.; Yasko, J. M. Drug Resistance. In Current Cancer Therapeutics, 2nd ed.;

51. Krasilnikov, O., V., Sabirov, R. Z., Ternovsky, V. I., Merzliak, P. G., and Muratkhodjaev, J. N. (1992) FEMS Microbiol. Immunol. 105, 93-100.

52. Krishnasastry, M., Walker, B., Braha, 0. & Bayley, H. (1994) Febs Lett. 356, 66-71.

53. Lejeune, A., Moorjani, M., Gicquaud, C., Lacroix, J., Poyet, P. &Gaudreault, R. C. (1994) Anticancer Res. 14, 915-920.

54. Lejeune, A., Poyet, P., & Gaudreault, R. C. (1997) Anticancer Res. 17, 3599-3606.

55. Liotta, L. A., Steeg, P. S., & Stetler-Stevenson, W. G. (1991) Cell 64, 327-336.

56. Lloyd, K. O., Larson, G., Stromberg, N., Thurin, J. & Karlson, K. A. (1983) Immunogenetics 17, 537-541.

57. Masuko, T., Yagita, H. and Hashimoto, Y. (1984) J. nat. Cancer Inst. 72, 523-530.

58. Menestrina, G. (1986) J. Membr. Biol. 90, 177-190.

59. Menzies, B. E. & Kemodle, D. S. (1994) Infect. Immun. 62, 1843-1847.

60. Moor, H. (1964) Histochem. 62, 546-580.

61. Moorjani, M., Lejeune, A., Gicquaud, C., Lacroix, J., Poyet, P., & Gaudreault, R. C. (1996) Anticancer Res. 16, 2831-2836.

68. New, R. R. C. Liposome: A Practical Approach, Oxford University Press, 1997: Chapter 2, 85-90; Chapter 6, 221-239.

62. Okada, M., & Natori, S. (1984) Biochem. J.222, 119-124.

63. Panchal, R. G., and Bayley H. (1995) J. Biol. Chem. 270, 23072-23076.

64. Panchal, R. G., Cusack, E., Cheley, S., & Bayley, H. (1996) Nature Biotechnology 14, 852-856.

65. Pastan, I. Chaudhary, V., & Fitzgerald, D. J. (1992) Annu.Rev. Biochem. 61, 331-354.

66. Pressman, D., and Korngold, L. (1953) Cancer (Phila.) 6, 619-623.

67. Rozencweig, M., Kenis, Atassi, G., Staquet, M., and Duarte-Karim, M. (1975) Cancer Chemother. Rep. 6, 131-136.

68. Sleytr, U. B., Sara, M., Messner, P., & Purn, D. (1994) J. Cell Biochem. 56, 171-176.

69. Thiesen, H.-J., Juhl, H. and Arndt, R. (1987) Cancer Res. 47, 419-423.

59. Tobkes, N., Wallace, B. A., & Bayley, H. (1995) Biochemistry 24, 1915-1920.

60. Wade, D., Boman, A., Wahlin, B., Drain, C. M., Andrea, D., Boman, H., & Merrifield, R. B. (1990) Proc. Natl. Acad. Sci. 87, 4761-4765.

61. Trouet, A. (1978) Eur. J Cancer 14, 105-111.

62. Ueda, R., Shiku, H., Pfreundschuh, M., Takahashi, T., Li, L. T. C., Whitmore, W. F., Jr., Oettgen, H. F. & Old, L. J. (1979) J. Exp. Med. 150, 564-579.

63. Walker, B. J., & Bayley, H.(1994) Protein Eng. 7, 91-97.

64. Walker, B. J., & Bayley, H.(1993) J. Biol. Chem. 268, 5285-5292.

65. Walker, B., Braha, O., Cheley, S., and Bayley, H. (1995) Chem. Biol. 2, 99-105.

66. Walker, B., Krishnasastry, M., Zom, L. & Bayley, H. (1994 a) Protein Eng. 7, 655-662.

67. Walker, B. J., Krishnasastry, M., Zorn, L., & Bayley, H. (1992) J. BioL Chem. 267, 21782-21786.

68. Walker, B. Walsh, N. & Bayley, H. (1994b) MRS Symp. Proc. 330, 209-215.

69. Ward, R. J, Palmer, M., Leonard, K. & Bhakdi, S. (1994) Biochemistry 33, 7477-7484.

67. Van Der Goot, F. G., Lakey, J., Pattius, F., Sorokine, C. M., Van Dorselear, A., & Buckey, J. T. (1992). Biochemistry 31, 8566-8570.

69. Yanchinski, S. Gen. Eng. News 1999, 19 (Jan 1), 7. Young, D. A., Prout, G. R., Jr., and Lin, C. W. (1985) Cancer

We claim:

1. A method for therapeutic treatment, comprising:
(a) contacting a cell membrane with a therapeutic composition that comprises an encapsulation vesicle and a pore forming agent on the surface of the encapsulation vesicle;
(b) allowing the cell membrane to incorporate the therapeutic composition so that the pore forming agent of the therapeutic composition may be activated by an activation condition.

2. The method of claim 1, wherein the pore forming agent comprises a biomolecule.

3. The method of claim 2, wherein the biomolecule comprises a protein.

4. The method of claim 3, wherein the protein is derived form a bacteria selected from the group consisting of α-hemolysin, E.coli hemolysin, E.coli colicin, B. thuringensis toxin, aerolysin, perfringolysin, pneumolysin, streptolysin O, and listeriolysin.

5. The method of claim 3, wherein the protein is a eucaryotic protein capable of lysing cells selected from the group consisting of defensin, magainin, complement, gramicidin, mellitin, perforin, yeast killer toxin and histolysin.

6. The method of claim 1, wherein the pore forming agent comprises a synthetic organic molecule.

7. The method of claim 3, wherein the protein is the α-hemolysin protein.

8. The method of claim 7, wherein the α-hemolysin protein is a mutant protein selected from the group consisting of R104C, E11C, K168C and D183C.

9. The method of claim 1, wherein the pore forming agent comprises a solid state material.

10. The method of claim 9, wherein the solid state material is selected from the group consisting of a zeolite, a nanotube, a nanorod, a nanocomposite, a nanowire, a nanodot, a quantum dot, a nanostructure, a plastic, a synthetic material, a silica material, a silicon material, an artificial bone material and a suicide inhibitor.

11. The method of claim 1, wherein the encapsulation vesicle is selected from the group consisting of a liposome, a liposphere, a stealth liposome, a nanoliposome, a nanoparticle, a micelle, a bubble, a microbubble, a microsphere, a nanosphere, a nanostructure, a microballoon, a microcapsule, an aerogel, a clathrate bound vesicle, a hexagonal structure, a cubic structure, a hexagonal II phase structure, and a nanoerythrosome.

12. The method of claim 1, wherein the encapsulation vesicle is selected from the group consisting of a T4 phage, an adenovirus, a polio virus, an influenza virus, an HIV virus, a bacteria, and a fungi.

13. The method of claim 1, wherein the encapsulation vesicle comprises a bioactive agent.

14. The method of claim 1, wherein the activation condition is endogenously provided by a targeted cell.

15. The method of claim 1, wherein the activation condition is exogenously applied by a source other than the targeted cell.

16. The method of claim 1, wherein the activation condition comprises a physical condition.

17. The method of claim 16, where the physical condition is selected from the group consisting of heat, light, and temperature change.

18. The method of claim 17, wherein the light is from a laser.

19. The method of claim 18, wherein the light causes a photodynamic effect.

20. The method of claim 1, wherein the activation condition comprises a chemical condition.

21. The method of claim 20, wherein the chemical condition is selected from the group consisting of changes in pH, changes in reduction potential, metal ions and protecting groups.

22. The method of claim 1, wherein the activation condition comprises a biochemical condition.

23. The method of claim 22, wherein the biochemical condition is from a biochemical substance selected from the group consisting of pathogenic cells, proteases, esterases, glycosidases, ectokinases and phoshpatases.

24. The method of claim 1, wherein the pore forming agent of the composition self assembles.

25. The method of claim 1, wherein the composition is manually assembled.

26. The method of claim 1, wherein the composition is assembled using covalent bonds.

27. The method of claim 1, wherein the composition is assembled using noncovalent bonds.

28. The method of claim 1, wherein the cell membrane incorporates the therapeutic composition by a fusion process.

29. The method of claim 1, wherein the cell membrane incorporates the therapeutic composition by a phagocytic process.

30. The method of claim 1, wherein the cell membrane is of a diseased cell.

31. The method of claim 30, wherein the diseased cell is a cancer cell.

32. The method of claim 30, where the diseased cell is a cell infected by the human immunodeficiency virus (HIV).

33. The method of claim 1, wherein the therapeutic composition further comprises a targeting ligand.

34. A method of therapeutic treatment that destroys a bioactive agent resistant diseased cell in a region of a patient after the cell membrane of the diseased cell has been contacted with a therapeutic composition, comprising:
(a) contacting the diseased cell with the therapeutic composition comprising an encapsulation vesicle, a pore forming agent on the surface of the encapsulation vesicle and an encapsulated bioactive agent;
(b) allowing the diseased cell to incorporate the therapeutic composition into the cell membrane by a fusion process so that the pore forming agent may be activated by an activation condition;
(c) delivering the bioactive agent into the interior of the diseased cell; and
(d) activating the pore forming agent by the activation condition to destroy the bioactive agent resistant diseased cell.
